(19) United States
(12) Patent Application Publication
(10) Pub. No.: US 2009/0301498 A1
(43) Pub. Date: Dec. 10, 2009

(54) ARRANGEMENT FOR SECURING A TEETH SUPPORT

(75) Inventor: Jan Christer Nordström, Stockholm (SE)

Correspondence Address: DRINKER BIDDLE & REATH (DC), 1500 K STREET, N.W., SUITE 1100, WASHINGTON, DC, 20005-1209, US

(73) Assignee: Jsn Christer NORDSTROM, Stockholm (SE)

(21) Appl. No.: 12/302,446

(22) PCT Filed: May 18, 2007
(86) PCT No.: PCT/SE07/50335

§ 371 Date: Jun. 11, 2009

(30) Foreign Application Priority Data
May 26, 2006 (SE) 0601168-8

(51) Int. Cl. A61F 5/56

(52) U.S. Cl. 128/848

(57) ABSTRACT

The present invention comprises an arrangement for securing a teeth support, in the form of a bite plate, in a predetermined position in relation to a person's arranged set of teeth and with said bite plate being adapted to carry a number of clamp devices, intended to fixedly but easily detachably be co-operable with one or more teeth, included in said set of teeth. Each one of said clamp devices has a retention bracket carried by the bite plate. A screw co-operates with a thread in the retention bracket and extends freely through the bite plate. One end portion of the screw is formed as a screw head and the other end potion thereof is adapted to present a tooth-adapted abutment surface. Said abutment surface is, by turning the screw in a first direction, pressable against a tooth surface for securing the bite plate and in relation to said tooth and, by turning the screw in a second opposite direction, loosenable from the tooth surface for a loosening of the bite plate.

### TECHNICAL FIELD OF THE INVENTION

The present invention generally relates to an arrangement for securing a teeth support in a predetermined position in relation to a person's arranged or related set of teeth and with said teeth support being adapted to carry a number of clamp devices, adapted to fixedly, but easily detachably, be co-operable with one or more teeth, included in said set of teeth.

The present invention gets a special application when the teeth support is given a form of a bite plate or splint.

Such a bite plate is adapted to be able to fix a mandible in a forward projected position in relation to a maxilla, thereby being able to provide conditions in order to somewhat forwardly advance the tongue and the root of the tongue.

A bite plate of the relevant type is utilized to eliminate or reduce snoring effects.

### BACKGROUND OF THE INVENTION

Methods, arrangements and designs, related to the above-mentioned technical field and nature, are previously known in a plurality of different embodiments.

When the special application of the present invention is as a bite plate, it is previously known to allow “custom-making” an acrylic bite plate from casts. Such a bite plate is adapted to temporarily be used during sleep, and it should then fix the mandible in a closed position and approx. 5 mm further forward than normally.

In doing so, the tongue attached by the root thereof on the inside of the mandible is advanced, and this gives space for a better and widened passage for the air behind the root of the tongue.

Bite plates of the relevant type normally consist of a plastic cast, having exposed U-shaped metallic brackets serving as clamping members, which are adapted to clamp themselves against one or more molars, both in the maxilla and the mandible, but which then leaves the canine and incisor teeth free.

Such a bite plate is, by means of the metallic brackets thereof, as clamping members, formed so that a patient can open the mouth by a force, the bite plate releasing the co-operation thereof with the molars.

### SUMMARY OF THE PRESENT INVENTION

### Technical Problem

If attention is paid to the circumstance that the technical considerations, which a person skilled in the relevant technical field has to do to be able to present a solution to one or more technical problems raised, are, on one hand, initially a necessary understanding of the measures and/or the sequence of measures that have to be taken, and on the other hand a necessary selection of the means that is/are required, then the subsequent technical problems would, in view of this, be relevant in the creation of the present subject matter of invention.

Therefore, in view of the prior art, as it has been described above, it should be seen as a technical problem to be capable of realizing the significance of, the advantages associated with and/or the technical measures and considerations that will be required to provide a clamping device between a bite plate and selected teeth of the mandible and maxilla and with the attachment force being easily adjustable, without needing to deflect previously known U-shaped spring elements mechanically.

There is a technical problem to be capable of realizing the significance of, the advantages associated with and/or the technical measures and considerations that will be required to allow said clamp device to have a retention bracket (securing bracket) carried by the bite plate, and to allow a screw to extend through this bracket, one end portion of said screw is formed as a screw head and the other end portion of said screw is adapted to present a tooth-adapted abutment surface.

There is a technical problem to be capable of realizing the significance of, the advantages associated with and/or the technical measures and considerations that will be required to allow said abutment surface, by turning the screw in a first direction, to be pressed or pressable against a selected tooth surface, such as the tooth surface of a molar, for securing the bracket and the bite plate.

There is a technical problem to be capable of realizing the significance of, the advantages associated with and/or the technical measures and considerations that will be required to allow a turning of the screw in a second opposite direction to provide conditions in order to loosen the bite plate from the impact against the tooth surface for an easy loosening of the bite plate.

There is a technical problem to be capable of realizing the significance of, the advantages associated with and/or the technical measures and considerations that will be required to allow the bite plate to be formed of plastic and said retention bracket to be entirely or partly contained within or attached to said bite plate.

There is a technical problem to be capable of realizing the significance of, the advantages associated with and/or the technical measures and considerations that will be required to allow a plate, having said tooth-adapted abutment surface, to be somewhat recessed in a notch in the bite plate.

There is a technical problem to be capable of realizing the significance of, the advantages associated with and/or the technical measures and considerations that will be required to allow said screw head to be co-ordinated with a notch in the bite plate and/or located near said retention bracket.

There is a technical problem to be capable of realizing the significance of, the advantages associated with and/or the technical measures and considerations that will be required to allow two (or four) clamp devices to be adapted to pressingly co-operate with a molar each, one in the maxilla as well as in the mandible.

There is a technical problem to be capable of realizing the significance of, the advantages associated with and/or the technical measures and considerations that will be required to allow the bite plate to be adapted to, by the two (or four) clamp devices thereof, allow to secure and/or locate the teeth of the mandible in an adapted and somewhat forwardly projected position, say approx. 5 mm.

### The Solution

The present invention is, in that connection, based on the known technique provided by way of introduction concerning an arrangement for securing a teeth support, in the form of a bite plate, in a predetermined position in relation to a person's arranged and related set of teeth and with said bite plate being adapted to carry a number of clamp devices, adapted to fixedly, but easily detachably, be co-operable with one or more teeth, included in said set of teeth.

In order to be able to solve one or more of the above-mentioned technical problems, the present invention particularly teaches that the known technique should be supplemented with allowing said clamp device to have a retention bracket carried by the bite plate, that a screw is threadly arranged through the retention bracket and freely through the bite plate, one end portion of which screw is formed as a screw head and the other end portion of which is adapted to present a tooth-adapted abutment surface.

Furthermore, the invention teaches that the abutment surface should, by turning the screw in a first direction, be pressable against the tooth surface for securing the bite plate in relation to said tooth and, by turning the screw in a second opposite direction, be loosenable from the tooth surface for a loosening of the bite plate.

As proposed embodiments, falling within the scope of the fundamental idea of the present invention, moreover it is taught that the bite plate should be formed of plastic, and that said retention bracket should be entirely or partly contained within said bite plate, and that said tooth-adapted abutment surface can be recessed in a notch in the bite plate.

Furthermore, it is taught that said screw head should be coordinated with a notch in the bite plate and/or located near the retention bracket.

Within the scope of the invention, there is also the possibility of allowing a clamp device each to be adapted to co-operate with a molar each, one in the maxilla as well as in the mandible within the left row of teeth and correspondingly in the right row of teeth.

The invention also teaches that the bite plate should be adapted to, by the clamp devices thereof, allow to secure the teeth of the mandible in a somewhat forwardly projected position, say approx. 5 mm.

### Advantages

The advantages that foremost may be regarded as characteristic of the present invention and the special significant features provided thereby are that, in this way, conditions have been created in order to be able to produce a bite plate and clamp devices that are coordinated with the bite plate and not only can offer an adjustable clamping force but furthermore can offer the possibility of easily removing or, alternatively, apply the bite plate for a temporary usage.

What foremost can be regarded as characteristic of the present invention is defined in the characterizing clause of the subsequent claim 1.

### BRIEF DESCRIPTION OF THE DRAWINGS

A presently proposed embodiment, having the significative features associated with the present invention, will now, for the purpose of exemplifying, be described more in detail, reference being made to the accompanying drawing, wherein:

FIG. 1 shows in a side view a person allotted a normal set of teeth,

FIG. 2 shows in a side view how a mandible, by means of a bite plate put in place, has been displaced forward a distance in relation to the maxilla,

FIG. 3 shows in a section a tooth with which a bite plate co-operates and one of four clamp devices, in accordance with the present invention,

FIG. 4 shows, in a somewhat enlarged view in relation to FIG. 3, the design of the clamp device according to the invention, and

FIG. 5 illustrates in a planar view and schematically the individual parts included in the clamp device.

### DESCRIPTION OF A PRESENTLY PROPOSED EMBODIMENT

It should then by way of introduction be emphasized that in the subsequent description of a presently proposed embodiment, which has the significative features associated with the invention and which is elucidated by the figures shown in the appended drawings, we have selected terms and a particular terminology with the intention to thereby primarily allow to make evident the inventive idea.

It should, however, in this connection be taken into consideration that expressions selected here should not be seen as limiting solely to the terms utilized and selected here, but it should be understood that each term selected in this manner should be interpreted so that it, in addition, comprises all technical equivalents operating in the same or substantially the same way, in order to, in this way, enable the achievement of the same or substantially the same intention and/or technical effect.

Thus, with a reference to the appended figures, the fundamental conditions for the present invention are shown schematically and in detail, and where the significative peculiarities or features associated with the invention have been made concrete, by the presently proposed embodiment described more in detail in the following.

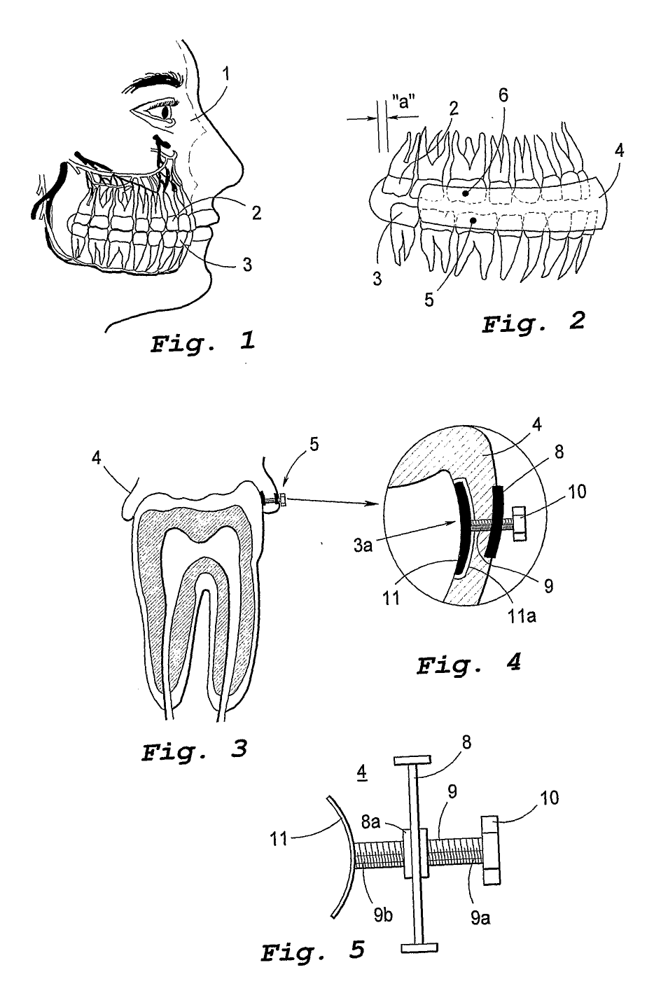

Thus, FIG. 1 allows illustrating the normal location of the normal set of teeth of a person 1.

Then, FIG. 1 illustrates a first set of teeth 2 of a maxilla and a second set of teeth 3 of a mandible, and where the individual teeth are coordinately illustrated.

Then, FIG. 2 illustrates more in detail how a bite plate 4 is placed between the upper row or set of teeth 2 and the lower row or set of teeth 3, while utilizing a first clamp device 5 located to the right and a second clamp device 6 located to the right, according to the provisions of the invention.

The invention also provides two clamp devices related to the left, which however are not shown.

The clamp device 5 and the clamp device 6 and other clamp devices are here identically formed, and therefore, for the purpose of simplification, the subsequent description will only comprise the clamp device 5.

The arrangement provided, according to the invention, is shown more in detail and described in connection with FIGS. 3, 4 and 5, and this arrangement 5 is adapted for securing a teeth support, in the form of a bite plate 4, in a predetermined position in relation to a person's arranged or related sets of teeth 2, 3 and with said bite plate 4 being adapted to carry a number of clamp devices 5, 6, here related to the right and adapted to fixedly, but easily detachably, be co-operable with one or more molars, included in said set of teeth 2, 3.

Normally, it is here a question of a co-operation with one or more molars.

The arrangement or the clamp device 5 has a retention bracket 8 carried by the bite plate 4 and attached to the bite plate 4, and utilizes a screw 9 co-ordinated with a threaded hole 8a, extending through the retention bracket 8 and with the screw 9 being arranged to pass freely through the bite plate 4.

One end portion 9a of the screw 9 is formed as a screw head 10, whereas the other end portion 9b of the screw is adapted to present a tooth-adapted abutment surface 11, such as a slightly calotte-shaped abutment surface.

Said abutment surface 11 is, by turning the screw 9 in a first direction, pressable against a tooth surface 3a for securing the bite plate 4 and in relation to said tooth, and is by turning the screw 9 in a second opposite direction, loosenable by the fact that the abutment surface 11 becomes loosenable from the tooth surface 3a for a loosening of the bite plate 4.

According to the invention, it is now taught that the bite plate 4 should be formed of plastic, and that said retention bracket 8 should be entirely or partly contained within, however attached to, said bite plate 4, according to FIG. 4.

Said tooth-adapted abutment surface 11 should advantageously be able to be recessed in a notch in the bite plate 4, where the notch in FIG. 4 has been given the reference designation 11a.

The screw head 10 could also be allowed to be coordinated with a notch in the bite plate 4 and/or located nearer the retention bracket 8 than what is illustrated in the accompanying drawing 5.

That is, more particularly, a first clamp device 5 is adapted to co-operate with one or more of the molars 3 in the mandible, whereas a second clamp device 6 is adapted to be co-operable with one or more of the molars 2 in the maxilla, while corresponding clamp devices are present on the other side.

Quite particularly, the bite plate 4 should, according to the present invention, be adapted to, by the clamp devices 5, 6 thereof, allow to secure the teeth of the mandible in a somewhat forwardly projected position, illustrated by a distance “a”, and where this distance should be selected to approx. 5 mm.

The invention is of course not limited to the embodiment given above as example, but may be subjected to modifications within the scope of the general idea according to the invention, illustrated in the subsequent claims.

Particularly, it should be taken into consideration that each unit and/or category shown can be combined with any other unit and/or category shown within the scope in order to be able to attain the desired technical function.

1. An arrangement for securing a teeth support, in the form of a bite plate, in a predetermined position in relation to a person's arranged or related set of teeth and with said bite plate being adapted to carry a number of clamp devices, adapted to fixedly but easily detachably be co-operable with one or more teeth, included in said set of teeth, characterized in that said clamp device has a retention bracket, carried by the bite plate, that a screw is arranged through said retention bracket and the bite plate, one end portion of which screw is formed as a screw head and the other end portion of which is adapted to present a tooth-adapted abutment surface, and that said abutment surface is, by turning the screw in a first direction, pressable against a tooth surface for securing the bite plate in relation to said tooth and, by turning the screw in a second opposite direction, loosenable from the tooth surface for a loosening of the bite plate.

2. Arrangement according to claim 1, characterized in that the bite plate is formed of plastic, and that said retention bracket is entirely or partly contained within, however attached to, said bite plate, and that said tooth-adapted abutment surface is recessed in a notch in the bite plate.

3. Arrangement according to claim 1 or 2, characterized in that said screw head is co-ordinated with a notch in the bite plate and/or located near the retention bracket.

4. Arrangement according to preceding claim 1, characterized in that a clamp device each is adapted to co-operate with a molar each, one in the maxilla as well as in the mandible.

5. Arrangement according to preceding claim 1, characterized in that the bite plate is adapted to, by the clamp devices thereof, allow to secure the teeth of the mandible in a somewhat forwardly projected position, say approx. 5 mm.
